SEQUENCE LISTING (121 sequences)

SEQ ID NO: 1 - DNA, 2133 bp, Homo Sapiens
gcggccgcgcgcactcgcacccgctgcccccgaggccctcctgcactctccccggcgccgctctccggccctcgccctgtccgccgccaccgccgccgccgccagagtcgccatgcagatcccgcgcgccgctcttctcccgctgctgctgctgctgctggcggcgcccgcctcggcgcagctgtcccgggccggccgctcggcgcctttggccgccgggtgcccagaccgctgcgagccggcgcgctgcccgccgcagccggagcactgcgagggcggccgggcccgggacgcgtgcggctgctgcgaggtgtgcggcgcgcccgagggcgccgcgtgcggcctgcaggagggcccgtgcggcgaggggctgcagtgcgtggtgcccttcggggtgccagcctcggccacggtgcggcggcgcgcgcaggccggcctctgtgtgtgcgccagcagcgagccggtgtgcggcagcgacgccaacacctacgccaacctgtgccagctgcgcgccgccagccgccgctccgagaggctgcaccggccgccggtcatcgtcctgcagcgcggagcctgcggccaagggcaggaagatcccaacagtttgcgccataaatataactttatcgcggacgtggtggagaagatcgcccctgccgtggttcatatcgaattgtttcgcaagcttccgttttctaaacgagaggtgccggtggctagtgggtctgggtttattgtgtcggaagatggactgatcgtgacaaatgcccacgtggtgaccaacaagcaccgggtcaaagttgagctgaagaacggtgccacttacgaagccaaaatcaaggatgtggatgagaaagcagacatcgcactcatcaaaattgaccaccagggcaagctgcctgtcctgctgcttggccgctcctcagagctgcggccgggagagttcgtggtcgccatcggaagcccgttttcccttcaaaacacagtcaccaccgggatcgtgagcaccacccagcgaggcggcaaagagctggggctccgcaactcagacatggactacatccagaccgacgccatcatcaactatggaaactcgggaggcccgttagtaaacctggacggtgaagtgattggaattaacactttgaaagtgacagctggaatctcctttgcaatcccatctgataagattaaaaagttcctcacggagtcccatgaccgacaggccaaaggaaaagccatcaccaagaagaagtatattggtatccgaatgatgtcactcacgtccagcaaagccaaagagctgaaggaccggcaccgggacttcccagacgtgatctcaggagcgtatataattgaagtaattcctgataccccagcagaagctggtggtctcaaggaaaacgacgtcataatcagcatcaatggacagtccgtggtctccgccaatgatgtcagcgacgtcattaaaagggaaagcaccctgaacatggtggtccgcaggggtaatgaagatatcatgatcacagtgattcccgaagaaattgacccataggcagaggcatgagctggacttcatgtttccctcaaagactctcccgtggatgacggatgaggactctgggctgctggaataggacactcaagacttttgactgccattttgtttgttcagtggagactccctggccaacagaatccttcttgatagtttgcaggcaaaacaaatgtaatgttgcagatccgcaggcagaagctctgcccttctgtatcctatgtatgcagtgtgctttttcttgccagcttgggccattcttgcttagacagtcagcatttgtctcctcctttaactgagtcatcatcttagtccaactaatgcagtcgatacaatgcgtagatagaagaagccccacgggagccaggatgggactggtcgtgtttgtgcttttctccaagtcagcacccaaaggtcaatgcacagagaccccgggtgggtgagcgctggcttctcaaacggccgaagttgcctcttttaggaatctctttggaattgggagcacgatgactctgagtttgagctattaaagtacttcttacacattgaaaaaaaaaaaaaaaaaaaaaaaaaaaaaa

SEQ ID NO: 2 - DNA, 19 bp, Homo sapiens
aggaagatcccaacagttt

SEQ ID NO: 3 - DNA, 19 bp, Homo sapiens
cccaacagtttgcgccata

SEQ ID NO: 4 - DNA, 19 bp, Homo Sapiens
ccaacagtttgcgccataa

SEQ ID NO: 5 - DNA, 19 bp, Homo Sapiens
caacagtttgcgccataaa

SEQ ID NO: 6 - DNA, 19 bp, Homo Sapiens
aacagtttgcgccataaat

SEQ ID NO: 7 - DNA, 19 bp, Homo Sapiens
acagtttgcgccataaata

SEQ ID NO: 8 - DNA, 19 bp, Homo Sapiens
cagtttgcgccataaatat

SEQ ID NO: 9 - DNA, 19 bp, Homo Sapiens
agtttgcgccataaatata

SEQ ID NO: 10 - DNA, 19 bp, Homo Sapiens
gtttgcgccataaatataa

SEQ ID NO: 11 - DNA, 19 bp, Homo Sapiens
tttgcgccataaatataac

SEQ ID NO: 12 - DNA, 19 bp, Homo Sapiens
ttgcgccataaatataact

SEQ ID NO: 13 - DNA, 19 bp, Homo Sapiens
tgcgccataaatataactt

SEQ ID NO: 14 - DNA, 19 bp, Homo Sapiens
gcgccataaatataacttt

SEQ ID NO: 15 - DNA, 19 bp, Homo Sapiens
cgccataaatataacttta

SEQ ID NO: 16 - DNA, 19 bp, Homo Sapiens
ataactttatcgcggacgt

SEQ ID NO: 17 - DNA, 19 bp, Homo Sapiens
tcgcggacgtggtggagaa

SEQ ID NO: 18 - DNA, 19 bp, Homo Sapiens
cggacgtggtggagaagat

SEQ ID NO: 19 - DNA, 19 bp, Homo Sapiens
tgccgtggttcatatcgaa

SEQ ID NO: 20 - DNA, 19 bp, Homo Sapiens
tggttcatatcgaattgtt

SEQ ID NO: 21 - DNA, 19 bp, Homo Sapiens
ggctagtgggtctgggttt

SEQ ID NO: 22 - DNA, 19 bp, Homo Sapiens
gctagtgggtctgggttta

SEQ ID NO: 23 - DNA, 19 bp, Homo Sapiens
ctagtgggtctgggtttat

SEQ ID NO: 24 - DNA, 19 bp, Homo Sapiens
tagtgggtctgggtttatt

SEQ ID NO: 25 - DNA, 19 bp, Homo Sapiens
gggtctgggtttattgtgt

SEQ ID NO: 26 - DNA, 19 bp, Homo Sapiens
ctgggtttattgtgtcgga

SEQ ID NO: 27 - DNA, 19 bp, Homo Sapiens
ggtttattgtgtcggaaga

SEQ ID NO: 28 - DNA, 19 bp, Homo Sapiens
gaagatggactgatcgtga

SEQ ID NO: 29 - DNA, 19 bp, Homo Sapiens
agatggactgatcgtgaca

SEQ ID NO: 30 - DNA, 19 bp, Homo Sapiens
atggactgatcgtgacaaa

SEQ ID NO: 31 - DNA, 19 bp, Homo Sapiens
ggactgatcgtgacaaatg

SEQ ID NO: 32 - DNA, 19 bp, Homo Sapiens
ccaacaagcaccgggtcaa

SEQ ID NO: 33 - DNA, 19 bp, Homo Sapiens
caacaagcaccgggtcaaa

SEQ ID NO: 34 - DNA, 19 bp, Homo Sapiens
acaagcaccgggtcaaagt

SEQ ID NO: 35 - DNA, 19 bp, Homo Sapiens
caagcaccgggtcaaagtt

SEQ ID NO: 36 - DNA, 19 bp, Homo Sapiens
agcaccgggtcaaagttga

SEQ ID NO: 37 - DNA, 19 bp, Homo Sapiens
gggtcaaagttgagctgaa

SEQ ID NO: 38 - DNA, 19 bp, Homo Sapiens
gtcaaagttgagctgaaga

SEQ ID NO: 39 - DNA, 19 bp, Homo Sapiens
tcaaagttgagctgaagaa

SEQ ID NO: 40 - DNA, 19 bp, Homo Sapiens
tgaagaacggtgccactta

SEQ ID NO: 41 - DNA, 19 bp, Homo Sapiens
gaagaacggtgccacttac

SEQ ID NO: 42 - DNA, 19 bp, Homo Sapiens
agaacggtgccacttacga

SEQ ID NO: 43 - DNA, 19 bp, Homo Sapiens
gaacggtgccacttacgaa

SEQ ID NO: 44 - DNA, 19 bp, Homo Sapiens
tgccacttacgaagccaaa

SEQ ID NO: 45 - DNA, 19 bp, Homo Sapiens
aaatcaaggatgtggatga

SEQ ID NO: 46 - DNA, 19 bp, Homo Sapiens
tcaaggatgtggatgagaa

SEQ ID NO: 47 - DNA, 19 bp, Homo Sapiens
caaggatgtggatgagaaa

SEQ ID NO: 48 - DNA, 19 bp, Homo Sapiens
ggatgtggatgagaaagca

SEQ ID NO: 49 - DNA, 19 bp, Homo Sapiens
gcagacatcgcactcatca

SEQ ID NO: 50 - DNA, 19 bp, Homo Sapiens
cagacatcgcactcatcaa

SEQ ID NO: 51 - DNA, 19 bp, Homo Sapiens
agacatcgcactcatcaaa

SEQ ID NO: 52 - DNA, 19 bp, Homo Sapiens
aaattgaccaccagggcaa

SEQ ID NO: 53 - DNA, 19 bp, Homo Sapiens
gcaactcagacatggacta

SEQ ID NO: 54 - DNA, 19 bp, Homo Sapiens
actcagacatggactacat

SEQ ID NO: 55 - DNA, 19 bp, Homo Sapiens
agaccgacgccatcatcaa

SEQ ID NO: 56 - DNA, 19 bp, Homo Sapiens
ccgacgccatcatcaacta

SEQ ID NO: 57 - DNA, 19 bp, Homo Sapiens
tagtaaacctggacggtga

SEQ ID NO: 58 - DNA, 19 bp, Homo Sapiens
cctggacggtgaagtgatt

SEQ ID NO: 59 - DNA, 19 bp, Homo Sapiens
acggtgaagtgattggaat

SEQ ID NO: 60 - DNA, 19 bp, Homo Sapiens
cggtgaagtgattggaatt

SEQ ID NO: 61 - DNA, 19 bp, Homo Sapiens
ggtgaagtgattggaatta

SEQ ID NO: 62 - DNA, 19 bp, Homo Sapiens
gtgaagtgattggaattaa

SEQ ID NO: 63 - DNA, 19 bp, Homo Sapiens
tgaaagtgacagctggaat

SEQ ID NO: 64 - DNA, 19 bp, Homo Sapiens
gaaagtgacagctggaatc

SEQ ID NO: 65 - DNA, 19 bp, Homo Sapiens
gctggaatctcctttgcaa

SEQ ID NO: 66 - DNA, 19 bp, Homo Sapiens
ttgcaatcccatctgataa

SEQ ID NO: 67 - DNA, 19 bp, Homo Sapiens
accgacaggccaaaggaaa

SEQ ID NO: 68 - DNA, 19 bp, Homo Sapiens
ccgacaggccaaaggaaaa

SEQ ID NO: 69 - DNA, 19 bp, Homo Sapiens
ggaaaagccatcaccaaga

SEQ ID NO: 70 - DNA, 19 bp, Homo Sapiens
gaaaagccatcaccaagaa

SEQ ID NO: 71 - DNA, 19 bp, Homo Sapiens
aagccatcaccaagaagaa

SEQ ID NO: 72 - DNA, 19 bp, Homo Sapiens
tcaccaagaagaagtatat

SEQ ID NO: 73 - DNA, 19 bp, Homo Sapiens
gaagaagtatattggtatc

SEQ ID NO: 74 - DNA, 19 bp, Homo Sapiens
tatattggtatccgaatga

SEQ ID NO: 75 - DNA, 19 bp, Homo Sapiens
cgtccagcaaagccaaaga

SEQ ID NO: 76 - DNA, 19 bp, Homo Sapiens
gcaaagccaaagagctgaa

SEQ ID NO: 77 - DNA, 19 bp, Homo Sapiens
ccaaagagctgaaggaccg

SEQ ID NO: 78 - DNA, 19 bp, Homo Sapiens
cgtgatctcaggagcgtat

SEQ ID NO: 79 - DNA, 19 bp, Homo Sapiens
tgatctcaggagcgtatat

SEQ ID NO: 80 - DNA, 19 bp, Homo Sapiens
tctcaggagcgtatataat

SEQ ID NO: 81 - DNA, 19 bp, Homo Sapiens
gagcgtatataattgaagt

SEQ ID NO: 82 - DNA, 19 bp, Homo Sapiens
gctggtggtctcaaggaaa

SEQ ID NO: 83 - DNA, 19 bp, Homo Sapiens
ctggtggtctcaaggaaaa

SEQ ID NO: 84 - DNA, 19 bp, Homo Sapiens
aggaaaacgacgtcataat

SEQ ID NO: 85 - DNA, 19 bp, Homo Sapiens
gacgtcataatcagcatca

SEQ ID NO: 86 - DNA, 19 bp, Homo Sapiens
acgtcataatcagcatcaa

SEQ ID NO: 87 - DNA, 19 bp, Homo Sapiens
cgtcataatcagcatcaat

SEQ ID NO: 88 - DNA, 19 bp, Homo Sapiens
atgtcagcgacgtcattaa

SEQ ID NO: 89 - DNA, 19 bp, Homo Sapiens
tgtcagcgacgtcattaaa

SEQ ID NO: 90 - DNA, 19 bp, Homo Sapiens
gggaaagcaccctgaacat

SEQ ID NO: 91 - DNA, 19 bp, Homo Sapiens
ccgcaggggtaatgaagat

SEQ ID NO: 92 - DNA, 19 bp, Homo Sapiens
atatcatgatcacagtgat

SEQ ID NO: 93 - DNA, 19 bp, Homo Sapiens
cagtgattcccgaagaaat

SEQ ID NO: 94 - DNA, 19 bp, Homo Sapiens
agtgattcccgaagaaatt

SEQ ID NO: 95 - DNA, 19 bp, Homo Sapiens
ccgaagaaattgacccata

SEQ ID NO: 96 - DNA, 19 bp, Homo Sapiens
ggcatgagctggacttcat

SEQ ID NO: 97 - DNA, 19 bp, Homo Sapiens
ttgatagtttgcaggcaaa

SEQ ID NO: 98 - DNA, 19 bp, Homo Sapiens
gcatttgtctcctccttta

SEQ ID NO: 99 - DNA, 19 bp, Homo Sapiens
catttgtctcctcctttaa

SEQ ID NO: 100 - DNA, 19 bp, Homo Sapiens
tcatcatcttagtccaact

SEQ ID NO: 101 - DNA, 19 bp, Homo Sapiens
catcatcttagtccaacta

SEQ ID NO: 102 - DNA, 19 bp, Homo Sapiens
atcatcttagtccaactaa

SEQ ID NO: 103 - DNA, 19 bp, Homo Sapiens
tcatcttagtccaactaat

SEQ ID NO: 104 - DNA, 19 bp, Homo Sapiens
aactaatgcagtcgataca

SEQ ID NO: 105 - DNA, 19 bp, Homo Sapiens
tcgatacaatgcgtagata

SEQ ID NO: 106 - DNA, 19 bp, Homo Sapiens
gatacaatgcgtagataga

SEQ ID NO: 107 - DNA, 19 bp, Homo Sapiens
caatgcgtagatagaagaa

SEQ ID NO: 108 - DNA, 19 bp, Homo Sapiens
ctctgagtttgagctatta

SEQ ID NO: 109 - DNA, 19 bp, Homo Sapiens
tctgagtttgagctattaa

SEQ ID NO: 110 - DNA, 19 bp, Homo Sapiens
ctgagtttgagctattaaa

SEQ ID NO: 111 - RNA, 19 nt, Artificial
  synthetic
gcuagugggucuggguuua

SEQ ID NO: 112 - RNA, 21 nt, Artificial
  synthetic
gcuagugggucuggguuuann

SEQ ID NO: 113 - RNA, 21 nt, Artificial
  synthetic
nncgaucacccagacccaaau

SEQ ID NO: 114 - RNA, 21 nt, Artificial
  synthetic
gcuagugggucuggguuuauu

SEQ ID NO: 115 - RNA, 21 nt, Artificial
  synthetic
uucgaucacccagacccaaau

SEQ ID NO: 116 - RNA, 19 nt, Artificial
  synthetic
gcuagugggucuggguuua

SEQ ID NO: 117 - RNA, 19 nt, Artificial
  synthetic
cgaucacccagacccaaau

SEQ ID NO: 118 - RNA, 48 nt, Artificial
  synthetic
gcuagugggucuggguuuannnnnnnnuaaacccagacccacuagcuu

SEQ ID NO: 119 - RNA, 25 nt, Artificial
  synthetic
gcuagugggucuggguuuauugugu

SEQ ID NO: 120 - RNA, 25 nt, Artificial
  synthetic
gcuagugggucuggguuuauugugu

SEQ ID NO: 121 - RNA, 27 nt, Artificial
  synthetic
uucgaucacccagacccaaauaacaca

(19) United States
(12) Patent Application Publication
(10) Pub. No.: US 2009/0012030 A1
(43) Pub. Date: Jan. 8, 2009

(54) RNAi-MEDIATED INHIBITIN OF HTRA1 FOR TREATMENT OF MACULAR DEGENERATION

(75) Inventors: Jon E. Chatterton, Crowley, TX (US); Martin B. Wax, Westlake, TX (US); Carmelo Romano, Fort Worth, TX (US); David P. Bingaman, Weatherford, TX (US)

Correspondence Address: ALCON, IP LEGAL, TB4-8, 6201 SOUTH FREEWAY, FORT WORTH, TX, 76134, US

(73) Assignee: ALCON RESEARCH, LTD., Fort Worth, TX (US)

(21) Appl. No.: 12/165,705

(22) Filed: Jul. 1, 2008

Related U.S. Application Data
(60) Provisional application No. 60/947,493, filed on Jul. 2, 2007.

(51) Int. Cl. A61K 31/7105; C07H 21/02; A61P 27/02

(52) U.S. Cl. 514/44; 536/24.5

(57) ABSTRACT

RNA interference is provided for inhibition of HTRA1 mRNA expression for treating patients with an HTRA1-mediated ocular disorder. In particular, methods are provided for treating age-related macular degeneration (AMD) and using interfering RNA molecules that attenuate expression of HTRA1 in patients having AMD or at risk of developing AMD.

### CLAIM FOR PRIORITY

The present application claims the benefit of co-pending U.S. Provisional Patent Application Ser. No. 60/947,493 filed Jul. 2, 2007.

### FIELD OF THE INVENTION

The present invention relates to the field of interfering RNA compositions for inhibition of function of HTRA1 in HTRA1-mediated ocular disorders, particularly for treatment of age-related macular degeneration (AMD) and for preventing progression of dry to wet AMD.

### BACKGROUND OF THE INVENTION

The human HTRA serine peptidase 1 (HTRA1) gene encodes a secreted serine protease, HtrA1, which exhibits a high degree of sequence similarity with the E. coli htrA (high temperature requirement) gene. HtrA1 contains an insulin-like growth factor (IGF) binding domain. It has been proposed to regulate IGF availability and cell growth (Zumbrunn and Trueb, 1996, FEBS Letters 398:189-192). HtrA1 expression is down-regulated in metastatic melanoma, and may thus indicate the degree of melanoma progression. Overexpression of HTRA1 in a metastatic melanoma cell line reduced proliferation and invasion in vitro, and reduced tumor growth in a xenograft mouse model (Baldi et al., 2002, Oncogene 21:6684-6688). HtrA1 expression is also down-regulated in ovarian cancer. In ovarian cancer cell lines, HtrA1 overexpression induces cell death, while antisense HTRA1 expression promoted anchorage-independent growth (Chien et al., 2004, Oncogene 23:1636-1644).

In addition to its effect on the IGF pathway, HtrA1 also inhibits signaling by the TGFβ family of growth factors (Oka et al., 2004, Development 131:1041-1053). HtrA1 can cleave amyloid precursor protein (APP), and HtrA1 inhibitors cause the accumulation of Aβ peptide in cultured cells. Thus, HtrA1 is also implicated in Alzheimer's disease (Grau et al., 2005, Proc. Natl. Acad. Sci. U.S.A. 102:6021-6026).

Recently, two groups identified a single nucleotide polymorphism (SNP) in the HTRA1 promoter region that dramatically increases the risk of developing wet age-related macular degeneration (AMD). The risk allele is associated with increased HTRA1 mRNA and protein expression, and HtrA1 is present in drusen in patients with AMD (Dewan et al., 2006, Science 314:989-992; Yang et al., 2006, Science 314:992-993).

Age-related macular degeneration (AMD) is the leading cause of irreversible loss of vision in people over the age of 65. With onset of AMD there is gradual loss of the light-sensitive photoreceptor cells in the back of the eye, the underlying pigment epithelial cells that support them metabolically, and the sharp central vision they provide. Age is the major risk factor for the onset of AMD: the likelihood of developing AMD triples after age 55. Smoking, light iris color, gender (women are at greater risk), obesity, and repeated exposure to UV radiation also increase the risk of AMD.

There are two forms of AMD: dry AMD and wet AMD. In dry AMD, drusen appear in the macula of the eye, the cells in the macula die, and vision becomes blurry. Dry AMD can progress in three stages: 1) early, 2) intermediate, and 3) advanced dry AMD. Dry AMD can also progress into wet AMD during any of these stages. Wet AMD (also known as exudative AMD), is associated with pathologic posterior segment neovascularization. The posterior segment neovascularization (PSNV) found in exudative AMD is characterized as pathologic choroidal neovascularization. Leakage from abnormal blood vessels forming in this process damages the macula and impairs vision, eventually leading to blindness.

Treatment strategies for wet AMD are few and palliative at best. Approved treatments for the PSNV in exudative AMD include laser photocoagulation and photodynamic therapy with Visudyne™; both therapies involve laser-induced occlusion of affected vasculature and are associated with localized laser-induced damage to the retina. Several different compounds are being evaluated clinically for the pharmacologic treatment of PSNV, including RETAANE™ (Alcon Research, Ltd.), adPEDF (GenVec), squalamine (Genaera), CA4P (OxiGENE), VEGF trap (Regeneron), anti-VEGF or VEGFR RNAi (Acuity and SIRNA, respectively), and LY333531 (Lilly). Lucentis™ (Genentech), an anti-VEGF antibody injected intravitreally, and Macugen™ (Eyetech/Pfizer), an anti-VEGF aptamer injected intravitreally, have recently been approved for such use.

Today, no pharmacologic therapy is approved for the treatment of macular edema associated with AMD. The current standard of care is laser photocoagulation, which is used to stabilize or resolve macular edema and retard the progression to later stage disease. Laser photocoagulation may reduce retinal ischemia by destroying healthy tissue and thereby decreasing metabolic demand; it also may modulate the expression and production of various cytokines and trophic factors.

There are no current treatments for preventing loss of vision after dry AMD enters an advanced stage. There are also no definitive methods for preventing progression of dry AMD to an advanced stage, other than by avoiding and/or reducing risk factors and using dietary supplements, which cannot guarantee or be relied on to stop AMD progression. Thus, there is a need for therapeutics that can treat dry AMD and prevent progression of dry to wet AMD.

As discussed above, HtrA1 is present in drusen in patients with AMD (Dewan et al., 2006, Science 314:989-992; Yang et al., 2006, Science 314:992-993). Drusen are already present in dry AMD patients. The presence of HtrA1 in drusen and increased HtrA1 expression in AMD patients, as well as the genetic studies discussed above, provide evidence that HtrA1 is a factor in AMD progression. Long-term reduction in HtrA1 expression using siRNAs targeting HTRA1 mRNAs is desirable for treating AMD and for reducing the risk of developing wet AMD.

### SUMMARY OF THE INVENTION

The invention provides interfering RNAs that silence HTRA serine peptidase 1 (HTRA1) mRNA expression, thus decreasing HTRA1 levels in patients with a HTRA1-mediated ocular disorder or at risk of developing a HTRA1-mediated ocular disorder. The interfering RNAs of the invention are useful for treating patients with HTRA1-mediated ocular disorders, particularly for patients with age-related macular degeneration (AMD), or for patients at risk of developing AMD.

The invention also provides a method of attenuating expression of a HTRA1 mRNA in a subject. In one aspect, the method comprises administering to the subject a composition comprising an effective amount of interfering RNA having a length of 19 to 49 nucleotides and a pharmaceutically acceptable carrier. In another aspect, administration is to an eye of the subject for attenuating expression of HTRA1 in a human.

In one aspect, the invention provides a method of attenuating expression of HTRA1 mRNA in an eye of a subject, comprising administering to the eye of the subject an interfering RNA that comprises a region that can recognize a portion of mRNA corresponding to SEQ ID NO: 1, which is the sense cDNA sequence encoding HTRA1 (GenBank Accession No. NM_{—}002775), wherein the expression of HTRA1 mRNA is attenuated thereby. In addition, the invention provides methods of treating an HTRA1-mediated ocular disorder in a subject in need thereof, comprising administering to the eye of the subject an interfering RNA that comprises a region that can recognize a portion of mRNA corresponding to a portion of SEQ ID NO: 1, wherein the expression of HTRA1 mRNA is attenuated thereby.

In certain aspects, an interfering RNA of the invention is designed to target an mRNA corresponding to a portion of SEQ ID NO: 1, wherein the portion comprises nucleotide 604, 612, 613, 614, 615, 616, 617, 618, 619, 620, 621, 622, 623, 624, 634, 643, 646, 671, 676, 731, 732, 733, 734, 738, 742, 745, 759, 761, 763, 765, 796, 797, 799, 800, 802, 808, 810, 811, 823, 824, 826, 827, 833, 850, 853, 854, 857, 873, 874, 875, 892, 1057, 1060, 1081, 1084, 1123, 1130, 1135, 1136, 1137, 1138, 1162, 1163, 1173, 1186, 1234, 1235, 1248, 1249, 1252, 1258, 1265, 1272, 1300, 1306, 1312, 1349, 1351, 1354, 1360, 1401, 1402, 1414, 1422, 1423, 1424, 1468, 1469, 1489, 1514, 1531, 1543, 1544, 1552, 1577, 1721, 1847, 1848, 1872, 1873, 1874, 1875, 1887, 1898, 1900, 1904, 2082, 2083, or 2084 of SEQ ID NO: 1. In particular aspects, a “portion of SEQ ID NO: 1” is about 19 to about 49 nucleotides in length.

In certain aspects, an interfering RNA of the invention has a length of about 19 to about 49 nucleotides. In other aspects, the interfering RNA comprises a sense nucleotide strand and an antisense nucleotide strand, wherein each strand has a region of at least near-perfect contiguous complementarity of at least 19 nucleotides with the other strand, and wherein the antisense strand can recognize a portion of HTRA1 mRNA corresponding to a portion of SEQ ID NO: 1, and has a region of at least near-perfect contiguous complementarity of at least 19 nucleotides with the portion of HTRA1 mRNA. The sense and antisense strands can be connected by a linker sequence, which allows the sense and antisense strands to hybridize to each other thereby forming a hairpin loop structure as described herein.

In still other aspects, an interfering RNA of the invention is a single-stranded interfering RNA, and wherein single-stranded interfering RNA recognizes a portion of mRNA corresponding to a portion of SEQ ID NO: 1. In certain aspects, the interfering RNA has a region of at least near-perfect contiguous complementarity of at least 19 nucleotides with the portion of mRNA corresponding to the portion of SEQ ID NO: 1. In other aspects, the portion of SEQ ID NO: 1 comprises nucleotide 604, 612, 613, 614, 615, 616, 617, 618, 619, 620, 621, 622, 623, 624, 634, 643, 646, 671, 676, 731, 732, 733, 734, 738, 742, 745, 759, 761, 763, 765, 796, 797, 799, 800, 802, 808, 810, 811, 823, 824, 826, 827, 833, 850, 853, 854, 857, 873, 874, 875, 892, 1057, 1060, 1081, 1084, 1123, 1130, 1135, 1136, 1137, 1138, 1162, 1163, 1173, 1186, 1234, 1235, 1248, 1249, 1252, 1258, 1265, 1272, 1300, 1306, 1312, 1349, 1351, 1354, 1360, 1401, 1402, 1414, 1422, 1423, 1424, 1468, 1469, 1489, 1514, 1531, 1543, 1544, 1552, 1577, 1721, 1847, 1848, 1872, 1873, 1874, 1875, 1887, 1898, 1900, 1904, 2082, 2083, or 2084 of SEQ ID NO: 1.

In still other aspects, an interfering RNA of the invention comprises: (a) a region of at least 13 contiguous nucleotides having at least 90% sequence complementarity to, or at least 90% sequence identity with, the penultimate 13 nucleotides of the 3′ end of a mRNA corresponding to any one of SEQ ID NO: 2-110; (b) a region of at least 14 contiguous nucleotides having at least 85% sequence complementarity to, or at least 85% sequence identity with, the penultimate 14 nucleotides of the 3′ end of an mRNA corresponding to any one of SEQ ID NO: 2-110; or (c) a region of at least 15, 16, 17, or 18 contiguous nucleotides having at least 80% sequence complementarity to, or at least 80% sequence identity with, the penultimate 15, 16, 17, or 18 nucleotides, respectively, of the 3′ end of an mRNA corresponding to any one of SEQ ID NO: 2-110; wherein the expression of the HTRA1 mRNA is attenuated thereby.

In further aspects, an interfering RNA of the invention or composition comprising an interfering RNA of the invention is administered to a subject via a topical, intravitreal, transcleral, periocular, conjunctival, subtenon, intracameral, subretinal, subconjunctival, retrobulbar, or intracanalicular route. The interfering RNA or composition can be administered, for example, via in vivo expression from an interfering RNA expression vector. In certain aspects, the interfering RNA or composition can be administered via an aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal route.

In one aspect, an interfering RNA molecule of the invention is isolated. The term “isolated” means that the interfering RNA is free of its total natural milieu.

The invention further provides methods of treating an HTRA1-mediated ocular disorder in a subject in need thereof, comprising administering to the subject a composition comprising a double-stranded siRNA molecule that down regulates expression of a HTRA1 gene via RNA interference, wherein each strand of the siRNA molecule is independently about 19 to about 27 nucleotides in length, and one strand of the siRNA molecule comprises a nucleotide sequence having substantial complementarity to an mRNA corresponding to the HTRA1 gene so that the siRNA molecule directs cleavage of the mRNA via RNA interference. In certain aspects, the siRNA molecule is administered via an aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal route.

The invention further provides for administering a second interfering RNA to a subject in addition to a first interfering RNA. The second interfering RNA may target the same mRNA target gene as the first interfering RNA or may target a different gene. Further, a third, fourth, or fifth, etc. interfering RNA may be administered in a similar manner.

Use of any of the embodiments as described herein in the preparation of a medicament for attenuating expression of HTRA1 mRNA is also an embodiment of the present invention.

Specific preferred embodiments of the invention will become evident from the following more detailed description of certain preferred embodiments and the claims.

### BRIEF DESCRIPTION OF THE DRAWINGS

FIG. 1 provides results of a qRT-PCR analysis of HTRA1 mRNA expression in HeLa cells transfected with HTRA1 siRNAs #1, #2, #3, and #4, each at 10 nM, 1 nM, and 0.1 nM.

### DETAILED DESCRIPTION OF THE INVENTION

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3^{rd} Edition or a dictionary known to those of skill in the art, such as the Oxford Dictionary of Biochemistry and Molecular Biology (Ed. Anthony Smith, Oxford University Press, Oxford, 2004).

As used herein, all percentages are percentages by weight, unless stated otherwise.

As used herein and unless otherwise indicated, the terms “a” and “an” are taken to mean “one”, “at least one” or “one or more”. Unless otherwise required by context, singular terms used herein shall include pluralities and plural terms shall include the singular.

In certain embodiments, the invention provides interfering RNA molecules that can direct cleavage and/or degradation of HTRA1 mRNA via RNA interference.

RNA interference (RNAi) is a process by which double-stranded RNA (dsRNA) is used to silence gene expression. While not wanting to be bound by theory, RNAi begins with the cleavage of longer dsRNAs into small interfering RNAs (siRNAs) by an RNaseIII-like enzyme, dicer. SiRNAs are dsRNAs that are usually about 19 to 28 nucleotides, or 20 to 25 nucleotides, or 21 to 22 nucleotides in length and often contain 2-nucleotide 3′ overhangs, and 5′ phosphate and 3′ hydroxyl termini. One strand of the siRNA is incorporated into a ribonucleoprotein complex known as the RNA-induced silencing complex (RISC). RISC uses this siRNA strand to identify mRNA molecules that are at least partially complementary to the incorporated siRNA strand, and then cleaves these target mRNAs or inhibits their translation. Therefore, the siRNA strand that is incorporated into RISC is known as the guide strand or the antisense strand. The other siRNA strand, known as the passenger strand or the sense strand, is eliminated from the siRNA and is at least partially homologous to the target mRNA. Those of skill in the art will recognize that, in principle, either strand of an siRNA can be incorporated into RISC and function as a guide strand. However, siRNA design (e.g., decreased siRNA duplex stability at the 5′ end of the desired guide strand) can favor incorporation of the desired guide strand into RISC.

The antisense strand of an siRNA is the active guiding agent of the siRNA in that the antisense strand is incorporated into RISC, thus allowing RISC to identify target mRNAs with at least partial complementarity to the antisense siRNA strand for cleavage or translational repression. RISC-mediated cleavage of mRNAs having a sequence at least partially complementary to the guide strand leads to a decrease in the steady state level of that mRNA and of the corresponding protein encoded by this mRNA. Alternatively, RISC can also decrease expression of the corresponding protein via translational repression without cleavage of the target mRNA.

Interfering RNAs of the invention appear to act in a catalytic manner for cleavage of target mRNA, i.e., interfering RNA is able to effect inhibition of target mRNA in substoichiometric amounts. As compared to antisense therapies, significantly less interfering RNA is required to provide a therapeutic effect under such cleavage conditions.

In certain embodiments, the invention provides methods of using interfering RNA to inhibit the expression of HTRA1 target mRNA thus decreasing HTRA1 levels in patients with a HTRA1-mediated ocular disorder. According to the present invention, interfering RNAs provided exogenously or expressed endogenously effect silencing of HTRA1 expression in ocular tissues.

The phrase, “attenuating expression of an mRNA,” as used herein, means administering or expressing an amount of interfering RNA (e.g., an siRNA) to reduce translation of the target mRNA into protein, either through mRNA cleavage or through direct inhibition of translation. The terms “inhibit,” “silencing,” and “attenuating” as used herein refer to a measurable reduction in expression of a target mRNA or the corresponding protein as compared with the expression of the target mRNA or the corresponding protein in the absence of an interfering RNA of the invention. The reduction in expression of the target mRNA or the corresponding protein is commonly referred to as “knock-down” and is reported relative to levels present following administration or expression of a non-targeting control RNA (e.g., a non-targeting control siRNA). Knock-down of expression of an amount including and between 50% and 100% is contemplated by embodiments herein. However, it is not necessary that such knock-down levels be achieved for purposes of the present invention.

Knock-down is commonly assessed by measuring the mRNA levels using quantitative polymerase chain reaction (qPCR) amplification or by measuring protein levels by western blot or enzyme-linked immunosorbent assay (ELISA). Analyzing the protein level provides an assessment of both mRNA cleavage as well as translation inhibition. Further techniques for measuring knock-down include RNA solution hybridization, nuclease protection, northern hybridization, gene expression monitoring with a microarray, antibody binding, radioimmunoassay, and fluorescence activated cell analysis.

Attenuating expression of HTRA1 by an interfering RNA molecule of the invention can be inferred in a human or other mammal by observing an improvement in symptoms of the disorder, including, for example, a slowing or reversal of vision loss that would indicate an inhibition of HTRA1.

The ability of interfering RNA to knock-down the levels of endogenous target gene expression in, for example, HeLa cells can be evaluated in vitro as follows. HeLa cells are plated 24 h prior to transfection in standard growth medium (e.g., DMEM supplemented with 10% fetal bovine serum). Transfection is performed using, for example, Dharmafect 1 (Dharmacon, Lafayette, Colo.) according to the manufacturer's instructions at interfering RNA concentrations ranging from 0.1 nM-100 nM. SiCONTROL™ Non-Targeting siRNA #1 and siCONTROL™ Cyclophilin B siRNA (Dharmacon) are used as negative and positive controls, respectively. Target mRNA levels and cyclophilin B mRNA (PPIB, NM_{—}000942) levels are assessed by qPCR 24 h post-transfection using, for example, a TAQMAN® Gene Expression Assay that preferably overlaps the target site (Applied Biosystems, Foster City, Calif.). The positive control siRNA gives essentially complete knockdown of cyclophilin B mRNA when transfection efficiency is 100%. Therefore, target mRNA knockdown is corrected for transfection efficiency by reference to the cyclophilin B mRNA level in cells transfected with the cyclophilin B siRNA. Target protein levels may be assessed approximately 72 h post-transfection (actual time dependent on protein turnover rate) by western blot, for example. Standard techniques for RNA and/or protein isolation from cultured cells are well-known to those skilled in the art. To reduce the chance of non-specific, off-target effects, the lowest possible concentration of interfering RNA is used that produces the desired level of knock-down in target gene expression. Human corneal epithelial cells or other human ocular cell lines may also be use for an evaluation of the ability of interfering RNA to knock-down levels of an endogenous target gene.

A number of animal models are known that can be used to test the activity of an interfering RNA molecule of the invention. For example, siRNA molecules can be tested in murine laser-induced models of choroidal neovascularization (CNV) as described in Reich et al., 2003, Mol. Vision 9:210-216; Shen et al., 2006, Gene Therapy 13:225-234; or Bora et al., 2006, J. Immunol. 177:1872-1878.

In one embodiment, a single interfering RNA targeting HTRA1 mRNA is administered to decrease HTRA1 levels. In other embodiments, two or more interfering RNAs targeting the HTRA1 mRNA are administered to decrease HTRA1 levels.

The GenBank database of the National Center for Biotechnology Information at ncbi.nlm.nih.gov provides the DNA sequence for HTRA1 as accession no. NM_{—}002775, provided in the “Sequence Listing” as SEQ ID NO: 1. SEQ ID NO: 1 provides the sense strand sequence of DNA that corresponds to the mRNA encoding HTRA1 (with the exception of “T” bases for “U” bases). The coding sequence for HTRA1 is from nucleotides 129 . . . 1571.

Equivalents of the above-cited HTRA1 mRNA sequence are alternative splice forms, allelic forms, isozymes, or a cognate thereof. A cognate is a HTRA1 mRNA from another mammalian species that is homologous to SEQ ID NO: 1.

In certain embodiments, a “subject” in need of treatment for a HTRA1-mediated ocular disorder or at risk for developing a HTRA1-mediated ocular disorder is a human or other mammal having a HTRA1-mediated ocular disorder or at risk of having a HTRA1-mediated ocular disorder associated with undesired or inappropriate expression or activity of targets as cited herein, i.e., HTRA1. For example, the subject has dry AMD or wet AMD, or has a risk of developing AMD (e.g. has a genetic risk factor for AMD). Ocular structures associated with such disorders may include the eye, retina, choroid, lens, cornea, trabecular meshwork, iris, optic nerve, optic nerve head, sclera, anterior or posterior segment, or ciliary body, for example. A subject may also be an ocular cell, cell culture, organ or an ex vivo organ or tissue or cell. In certain embodiments, a subject has dry AMD, and the methods of the invention can prevent progression of dry to wet AMD. In other embodiments, a subject has dry AMD, and the methods of the invention cause improvement of symptoms (e.g. improve vision), and can prevent further vision loss.

“HTRA1-mediated ocular disorder,” as used herein, means conditions of the eye where HTRA1 mediates the condition, particularly in age-related macular degeneration (AMD). A HTRA1-mediated ocular disorder includes conditions such as wet AMD and dry AMD.

The term “siRNA” as used herein refers to a double-stranded interfering RNA unless otherwise noted. Typically, an siRNA of the invention is a double-stranded nucleic acid molecule comprising two nucleotide strands, each strand having about 19 to about 28 nucleotides (i.e. about 19, 20, 21, 22, 23, 24, 25, 26, 27, or 28 nucleotides). The phrase “interfering RNA having a length of 19 to 49 nucleotides” when referring to a double-stranded interfering RNA means that the antisense and sense strands independently have a length of about 19 to about 49 nucleotides, including interfering RNA molecules where the sense and antisense strands are connected by a linker molecule.

In addition to siRNA molecules, other interfering RNA molecules and RNA-like molecules can interact with RISC and silence gene expression. Examples of other interfering RNA molecules that can interact with RISC include short hairpin RNAs (shRNAs), single-stranded siRNAs, microRNAs (miRNAs), and dicer-substrate 27-mer duplexes. Examples of RNA-like molecules that can interact with RISC include siRNA, single-stranded siRNA, microRNA, and shRNA molecules containing one or more chemically modified nucleotides, one or more non-nucleotides, one or more deoxyribonucleotides, and/or one or more non-phosphodiester linkages. All RNA or RNA-like molecules that can interact with RISC and participate in RISC-mediated changes in gene expression are referred to herein as “interfering RNAs” or “interfering RNA molecules.” SiRNAs, single-stranded siRNAs, shRNAs, miRNAs, and dicer-substrate 27-mer duplexes are, therefore, subsets of “interfering RNAs” or “interfering RNA molecules.”

Single-stranded interfering RNA has been found to effect mRNA silencing, albeit less efficiently than double-stranded RNA. Therefore, embodiments of the present invention also provide for administration of a single-stranded interfering RNA that has a region of at least near-perfect contiguous complementarity with a portion of SEQ ID NO: 1. The single-stranded interfering RNA has a length of about 19 to about 49 nucleotides as for the double-stranded interfering RNA cited above. The single-stranded interfering RNA has a 5′ phosphate or is phosphorylated in situ or in vivo at the 5′ position. The term “5′ phosphorylated” is used to describe, for example, polynucleotides or oligonucleotides having a phosphate group attached via ester linkage to the C5 hydroxyl of the sugar (e.g., ribose, deoxyribose, or an analog of same) at the 5′ end of the polynucleotide or oligonucleotide.

Single-stranded interfering RNAs can be synthesized chemically or by in vitro transcription or expressed endogenously from vectors or expression cassettes as described herein in reference to double-stranded interfering RNAs. 5′ Phosphate groups may be added via a kinase, or a 5′ phosphate may be the result of nuclease cleavage of an RNA. A hairpin interfering RNA is a single molecule (e.g., a single oligonucleotide chain) that comprises both the sense and antisense strands of an interfering RNA in a stem-loop or hairpin structure (e.g., a shRNA). For example, shRNAs can be expressed from DNA vectors in which the DNA oligonucleotides encoding a sense interfering RNA strand are linked to the DNA oligonucleotides encoding the reverse complementary antisense interfering RNA strand by a short spacer. If needed for the chosen expression vector, 3′ terminal T's and nucleotides forming restriction sites may be added. The resulting RNA transcript folds back onto itself to form a stem-loop structure.

Nucleic acid sequences cited herein are written in a 5′ to 3′ direction unless indicated otherwise. The term “nucleic acid,” as used herein, refers to either DNA or RNA or a modified form thereof comprising the purine or pyrimidine bases present in DNA (adenine “A,” cytosine “C,” guanine “G,” thymine “T”) or in RNA (adenine “A,” cytosine “C,” guanine “G,” uracil “U”). Interfering RNAs provided herein may comprise “T” bases, particularly at 3′ ends, even though “T” bases do not naturally occur in RNA. “Nucleic acid” includes the terms “oligonucleotide” and “polynucleotide” and can refer to a single-stranded molecule or a double-stranded molecule. A double-stranded molecule is formed by Watson-Crick base pairing between A and T bases, C and G bases, and between A and U bases. The strands of a double-stranded molecule may have partial, substantial or full complementarity to each other and will form a duplex hybrid, the strength of bonding of which is dependent upon the nature and degree of complementarity of the sequence of bases.

The phrase “DNA target sequence” as used herein refers to the DNA sequence that is used to derive an interfering RNA of the invention. The phrases “RNA target sequence,” “interfering RNA target sequence,” and “RNA target” as used herein refer to the HTRA1 mRNA or the portion of the HTRA1 mRNA sequence that can be recognized by an interfering RNA of the invention, whereby the interfering RNA can silence HTRA1 gene expression as discussed herein. An “RNA target sequence,” an “siRNA target sequence,” and an “RNA target” are typically mRNA sequences that correspond to a portion of a DNA sequence. An mRNA sequence is readily deduced from the sequence of the corresponding DNA sequence. For example, SEQ ID NO: 1 provides the sense strand sequence of DNA corresponding to the mRNA for HTRA1. The mRNA sequence is identical to the DNA sense strand sequence with the “T” bases replaced with “U” bases. Therefore, the mRNA sequence of HTRA1 is known from SEQ ID NO: 1. A target sequence in the mRNAs corresponding to SEQ ID NO: 1 may be in the 5′ or 3′ untranslated regions of the mRNA as well as in the coding region of the mRNA.

In certain embodiments, interfering RNA target sequences (e.g., siRNA target sequences) within a target mRNA sequence are selected using available design tools. Interfering RNAs corresponding to a HTRA1 target sequence are then tested in vitro by transfection of cells expressing the target mRNA followed by assessment of knockdown as described herein. The interfering RNAs can be further evaluated in vivo using animal models as described herein.

Techniques for selecting target sequences for siRNAs are provided, for example, by Tuschl, T. et al., “The siRNA User Guide,” revised May 6, 2004, available on the Rockefeller University web site; by Technical Bulletin #506, “siRNA Design Guidelines,” Ambion Inc. at Ambion's web site; and by other web-based design tools at, for example, the Invitrogen, Dharmacon, Integrated DNA Technologies, Genscript, or Proligo web sites. Initial search parameters can include G/C contents between 35% and 55% and siRNA lengths between 19 and 27 nucleotides. The target sequence may be located in the coding region or in the 5′ or 3′ untranslated regions of the mRNA. The target sequences can be used to derive interfering RNA molecules, such as those described herein.

Table 1 lists examples of HTRA1 DNA target sequences of SEQ ID NO: 1 from which interfering RNA molecules of the present invention are designed in a manner as set forth above.

TABLE 1

HTRA1 Target Sequences for siRNAs

| | | |
|---|---|---|
| | # of Starting | |
| | Nucleotide | |
| | with | |
| HTRA1 Target | reference to | |
| Sequence | SEQ ID NO: 1 | SEQ ID NO: |

| | | |
|---|---|---|
| AGGAAGATCCCAACAGTTT | 604 | 2 |
| CCCAACAGTTTGCGCCATA | 612 | 3 |
| CCAACAGTTTGCGCCATAA | 613 | 4 |
| CAACAGTTTGCGCCATAAA | 614 | 5 |
| AACAGTTTGCGCCATAAAT | 615 | 6 |
| ACAGTTTGCGCCATAAATA | 616 | 7 |
| CAGTTTGCGCCATAAATAT | 617 | 8 |
| AGTTTGCGCCATAAATATA | 618 | 9 |
| GTTTGCGCCATAAATATAA | 619 | 10 |
| TTTGCGCCATAAATATAAC | 620 | 11 |
| TTGCGCCATAAATATAACT | 621 | 12 |
| TGCGCCATAAATATAACTT | 622 | 13 |
| GCGCCATAAATATAACTTT | 623 | 14 |
| CGCCATAAATATAACTTTA | 624 | 15 |
| ATAACTTTATCGCGGACGT | 634 | 16 |
| TCGCGGACGTGGTGGAGAA | 643 | 17 |
| CGGACGTGGTGGAGAAGAT | 646 | 18 |
| TGCCGTGGTTCATATCGAA | 671 | 19 |
| TGGTTCATATCGAATTGTT | 676 | 20 |
| GGCTAGTGGGTCTGGGTTT | 731 | 21 |
| GCTAGTGGGTCTGGGTTTA | 732 | 22 |
| CTAGTGGGTCTGGGTTTAT | 733 | 23 |
| TAGTGGGTCTGGGTTTATT | 734 | 24 |
| GGGTCTGGGTTTATTGTGT | 738 | 25 |
| CTGGGTTTATTGTGTCGGA | 742 | 26 |
| GGTTTATTGTGTCGGAAGA | 745 | 27 |
| GAAGATGGACTGATCGTGA | 759 | 28 |
| AGATGGACTGATCGTGACA | 761 | 29 |
| ATGGACTGATCGTGACAAA | 763 | 30 |
| GGACTGATCGTGACAAATG | 765 | 31 |
| CCAACAAGCACCGGGTCAA | 796 | 32 |
| CAACAAGCACCGGGTCAAA | 797 | 33 |
| ACAAGCACCGGGTCAAAGT | 799 | 34 |
| CAAGCACCGGGTCAAAGTT | 800 | 35 |
| AGCACCGGGTCAAAGTTGA | 802 | 36 |
| GGGTCAAAGTTGAGCTGAA | 808 | 37 |
| GTCAAAGTTGAGCTGAAGA | 810 | 38 |
| TCAAAGTTGAGCTGAAGAA | 811 | 39 |
| TGAAGAACGGTGCCACTTA | 823 | 40 |
| GAAGAACGGTGCCACTTAC | 824 | 41 |
| AGAACGGTGCCACTTACGA | 826 | 42 |
| GAACGGTGCCACTTACGAA | 827 | 43 |
| TGCCACTTACGAAGCCAAA | 833 | 44 |
| AAATCAAGGATGTGGATGA | 850 | 45 |
| TCAAGGATGTGGATGAGAA | 853 | 46 |
| CAAGGATGTGGATGAGAAA | 854 | 47 |
| GGATGTGGATGAGAAAGCA | 857 | 48 |
| GCAGACATCGCACTCATCA | 873 | 49 |
| CAGACATCGCACTCATCAA | 874 | 50 |
| AGACATCGCACTCATCAAA | 875 | 51 |
| AAATTGACCACCAGGGCAA | 892 | 52 |
| GCAACTCAGACATGGACTA | 1057 | 53 |
| ACTCAGACATGGACTACAT | 1060 | 54 |
| AGACCGACGCCATCATCAA | 1081 | 55 |
| CCGACGCCATCATCAACTA | 1084 | 56 |
| TAGTAAACCTGGACGGTGA | 1123 | 57 |
| CCTGGACGGTGAAGTGATT | 1130 | 58 |
| ACGGTGAAGTGATTGGAAT | 1135 | 59 |
| CGGTGAAGTGATTGGAATT | 1136 | 60 |
| GGTGAAGTGATTGGAATTA | 1137 | 61 |
| GTGAAGTGATTGGAATTAA | 1138 | 62 |
| TGAAAGTGACAGCTGGAAT | 1162 | 63 |
| GAAAGTGACAGCTGGAATC | 1163 | 64 |
| GCTGGAATCTCCTTTGCAA | 1173 | 65 |
| TTGCAATCCCATCTGATAA | 1186 | 66 |
| ACCGACAGGCCAAAGGAAA | 1234 | 67 |
| CCGACAGGCCAAAGGAAAA | 1235 | 68 |
| GGAAAAGCCATCACCAAGA | 1248 | 69 |
| GAAAAGCCATCACCAAGAA | 1249 | 70 |
| AAGCCATCACCAAGAAGAA | 1252 | 71 |
| TCACCAAGAAGAAGTATAT | 1258 | 72 |
| GAAGAAGTATATTGGTATC | 1265 | 73 |
| TATATTGGTATCCGAATGA | 1272 | 74 |
| CGTCCAGCAAAGCCAAAGA | 1300 | 75 |
| GCAAAGCCAAAGAGCTGAA | 1306 | 76 |
| CCAAAGAGCTGAAGGACCG | 1312 | 77 |
| CGTGATCTCAGGAGCGTAT | 1349 | 78 |
| TGATCTCAGGAGCGTATAT | 1351 | 79 |
| TCTCAGGAGCGTATATAAT | 1354 | 80 |
| GAGCGTATATAATTGAAGT | 1360 | 81 |
| GCTGGTGGTCTCAAGGAAA | 1401 | 82 |
| CTGGTGGTCTCAAGGAAAA | 1402 | 83 |
| AGGAAAACGACGTCATAAT | 1414 | 84 |
| GACGTCATAATCAGCATCA | 1422 | 85 |
| ACGTCATAATCAGCATCAA | 1423 | 86 |
| CGTCATAATCAGCATCAAT | 1424 | 87 |
| ATGTCAGCGACGTCATTAA | 1468 | 88 |
| TGTCAGCGACGTCATTAAA | 1469 | 89 |
| GGGAAAGCACCCTGAACAT | 1489 | 90 |
| CCGCAGGGGTAATGAAGAT | 1514 | 91 |
| ATATCATGATCACAGTGAT | 1531 | 92 |
| CAGTGATTCCCGAAGAAAT | 1543 | 93 |
| AGTGATTCCCGAAGAAATT | 1544 | 94 |
| CCGAAGAAATTGACCCATA | 1552 | 95 |
| GGCATGAGCTGGACTTCAT | 1577 | 96 |
| TTGATAGTTTGCAGGCAAA | 1721 | 97 |
| GCATTTGTCTCCTCCTTTA | 1847 | 98 |
| CATTTGTCTCCTCCTTTAA | 1848 | 99 |
| TCATCATCTTAGTCCAACT | 1872 | 100 |
| CATCATCTTAGTCCAACTA | 1873 | 101 |
| ATCATCTTAGTCCAACTAA | 1874 | 102 |
| TCATCTTAGTCCAACTAAT | 1875 | 103 |
| AACTAATGCAGTCGATACA | 1887 | 104 |
| TCGATACAATGCGTAGATA | 1898 | 105 |
| GATACAATGCGTAGATAGA | 1900 | 106 |
| CAATGCGTAGATAGAAGAA | 1904 | 107 |
| CTCTGAGTTTGAGCTATTA | 2082 | 108 |
| TCTGAGTTTGAGCTATTAA | 2083 | 109 |
| CTGAGTTTGAGCTATTAAA | 2084 | 110 |

As cited in the examples above, one of skill in the art is able to use the target sequence information provided in Table 1 to design interfering RNAs having a length shorter or longer than the sequences provided in Table 1 by referring to the sequence position in SEQ ID NO: 1 and adding or deleting nucleotides complementary or near complementary to SEQ ID NO: 1.

For example, SEQ ID NO: 22 represents an example of a 19-nucleotide DNA target sequence for HTRA1 mRNA, and is present at nucleotides 732 to 750 of SEQ ID NO: 1:

| | |
|---|---|
| 5′- GCUAGUGGGUCUGGGUUUA -3′. | SEQ ID NO: 111 |

An example of an siRNA of the invention for targeting a corresponding mRNA sequence of SEQ ID NO: 22 and having 21-nucleotide strands and a 2-nucleotide 3′ overhang is:

| | |
|---|---|
| 5′- GCUAGUGGGUCUGGGUUUANN -3′ | SEQ ID NO: 112 |
| 3′- NNCGAUCACCCAGACCCAAAU -5′. | SEQ ID NO: 113 |

Each “N” residue can be any nucleotide (A, C, G, U, or T) or a modified nucleotide. The 3′ end can have a number of “N” residues between and including 1, 2, 3, 4, 5, and 6. The “N” residues on either strand can be the same residue (e.g., UU, AA, CC, GG, or TT) or they can be different (e.g., AC, AG, AU, CA, CG, CU, GA, GC, GU, UA, UC, or UG). The 3′ overhangs can be the same or they can be different. In one embodiment, both strands have a 3′UU overhang.

An example of an siRNA of the invention for targeting a corresponding mRNA sequence of SEQ ID NO: 22 and having 21-nucleotide strands and a 3′UU overhang on each strand is:

| | |
|---|---|
| 5′- GCUAGUGGGUCUGGGUUUAUU -3′ | SEQ ID NO: 114 |
| 3′- UUCGAUCACCCAGACCCAAAU -5′. | SEQ ID NO: 115 |

The interfering RNA may have a 5′ overhang of nucleotides or it may have blunt ends. An siRNA of the invention for targeting a corresponding mRNA sequence of SEQ ID NO: 22 and having 19-nucleotide strands and blunt ends is:

| | |
|---|---|
| 5′- GCUAGUGGGUCUGGGUUUA -3′ | SEQ ID NO: 116 |
| 3′- CGAUCACCCAGACCCAAAU -5′. | SEQ ID NO: 117 |

The strands of a double-stranded interfering RNA (e.g., an siRNA) may be connected by a linker sequence to form a hairpin or stem-loop structure (e.g., an shRNA). An example of an shRNA of the invention targeting a corresponding mRNA sequence of SEQ ID NO: 22 and having a 19 bp double-stranded stem region and a 3′UU overhang is:

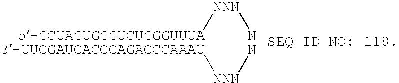

N is a nucleotide A, T, C, G, U, or a modified form known by one of ordinary skill in the art. The number of nucleotides N in the loop (i.e. linker sequence) is between and including 3 to 23, or 5 to 15, or 7 to 13, or 4 to 9, or 9 to 11. In certain embodiments, the number of nucleotides in a linker sequence is about 9. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

The siRNA target sequence identified above can be extended at the 3′ end to facilitate the design of dicer-substrate 27-mer duplexes. Extension of the 19-nucleotide DNA target sequence (SEQ ID NO: 22) identified in the HTRA1 DNA sequence (SEQ ID NO: 1) by 6 nucleotides yields a 25-nucleotide DNA target sequence present at nucleotides 732 to 756 of SEQ ID NO: 1:

| | |
|---|---|
| 5′- GCUAGUGGGUCUGGGUUUAUUGUGU -3′. | SEQ ID NO: 119 |

An example of a dicer-substrate 27-mer duplex of the invention for targeting a corresponding mRNA sequence of SEQ ID NO: 22 is:

| | |
|---|---|
| 5′- GCUAGUGGGUCUGGGUUUAUUGUGU -3′ | SEQ ID NO: 120 |
| 3′- UUCGAUCACCCAGACCCAAAUAACACA -5′. | SEQ ID NO: 121 |

The two nucleotides at the 3′ end of the sense strand (i.e., the GU nucleotides of SEQ ID NO: 120) may be deoxynucleotides for enhanced processing. Design of dicer-substrate 27-mer duplexes from 19-21 nucleotide target sequences, such as provided herein, is further discussed by the Integrated DNA Technologies (IDT) website and by Kim, D.-H. et al., (February, 2005) Nature Biotechnology 23:2; 222-226.

The target RNA cleavage reaction guided by siRNAs and other forms of interfering RNA is highly sequence specific. For example, in general, an siRNA molecule contains a sense nucleotide strand identical in sequence to a portion of the target mRNA and an antisense nucleotide strand exactly complementary to a portion of the target for inhibition of mRNA expression. However, 100% sequence complementarity between the antisense siRNA strand and the target mRNA, or between the antisense siRNA strand and the sense siRNA strand, is not required to practice the present invention, so long as the interfering RNA can recognize the target mRNA and silence expression of the HTRA1 gene. Thus, for example, the invention allows for sequence variations between the antisense strand and the target mRNA and between the antisense strand and the sense strand, including nucleotide substitutions that do not affect activity of the interfering RNA molecule, as well as variations that might be expected due to genetic mutation, strain polymorphism, or evolutionary divergence, wherein the variations do not preclude recognition of the antisense strand to the target mRNA.

In one embodiment of the invention, interfering RNA of the invention has a sense strand and an antisense strand, and the sense and antisense strands comprise a region of at least near-perfect contiguous complementarity of at least 19 nucleotides. In another embodiment of the invention, an interfering RNA of the invention has a sense strand and an antisense strand, and the antisense strand comprises a region of at least near-perfect contiguous complementarity of at least 19 nucleotides to a target sequence of HTRA1 mRNA, and the sense strand comprises a region of at least near-perfect contiguous identity of at least 19 nucleotides with a target sequence of HTRA1 mRNA, respectively. In a further embodiment of the invention, the interfering RNA comprises a region of at least 13, 14, 15, 16, 17, or 18 contiguous nucleotides having percentages of sequence complementarity to or, having percentages of sequence identity with, the penultimate 13, 14, 15, 16, 17, or 18 nucleotides, respectively, of the 3′ end of the corresponding target sequence within an mRNA. The length of each strand of the interfering RNA comprises about 19 to about 49 nucleotides, and may comprise a length of about 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides.

In certain embodiments, the antisense strand of an interfering RNA of the invention has at least near-perfect contiguous complementarity of at least 19 nucleotides with the target mRNA. “Near-perfect,” as used herein, means the antisense strand of the siRNA is “substantially complementary to,” and the sense strand of the siRNA is “substantially identical to” at least a portion of the target mRNA. “Identity,” as known by one of ordinary skill in the art, is the degree of sequence relatedness between nucleotide sequences as determined by matching the order and identity of nucleotides between the sequences. In one embodiment, the antisense strand of an siRNA having 80% and between 80% up to 100% complementarity, for example, 85%, 90% or 95% complementarity, to the target mRNA sequence are considered near-perfect complementarity and may be used in the present invention. “Perfect” contiguous complementarity is standard Watson-Crick base pairing of adjacent base pairs. “At least near-perfect” contiguous complementarity includes “perfect” complementarity as used herein. Computer methods for determining identity or complementarity are designed to identify the greatest degree of matching of nucleotide sequences, for example, BLASTN (Altschul, S. F., et al. (1990) J. Mol. Biol. 215:403-410).

The term “percent identity” describes the percentage of contiguous nucleotides in a first nucleic acid molecule that is the same as in a set of contiguous nucleotides of the same length in a second nucleic acid molecule. The term “percent complementarity” describes the percentage of contiguous nucleotides in a first nucleic acid molecule that can base pair in the Watson-Crick sense with a set of contiguous nucleotides in a second nucleic acid molecule.

The relationship between a target mRNA and one strand of an siRNA (the sense strand) is that of identity. The sense strand of an siRNA is also called a passenger strand, if present. The relationship between a target mRNA and the other strand of an siRNA (the antisense strand) is that of complementarity. The antisense strand of an siRNA is also called a guide strand.

There may be a region or regions of the antisense siRNA strand that is (are) not complementary to a portion of SEQ ID NO: 1. Non-complementary regions may be at the 3′, 5′ or both ends of a complementary region or between two complementary regions. A region can be one or more bases.

The sense and antisense strands in an interfering RNA molecule can also comprise nucleotides that do not form base pairs with the other strand. For example, one or both strands can comprise additional nucleotides or nucleotides that do not pair with a nucleotide in that position on the other strand, such that a bulge or a mismatch is formed when the strands are hybridized. Thus, an interfering RNA molecule of the invention can comprise sense and antisense strands having mismatches, G-U wobbles, or bulges. Mismatches, G-U wobbles, and bulges can also occur between the antisense strand and its target (see, for example, Saxena et al., 2003, J. Biol. Chem. 278:44312-9).

One or both of the strands of double-stranded interfering RNA may have a 3′ overhang of from 1 to 6 nucleotides, which may be ribonucleotides or deoxyribonucleotides or a mixture thereof. The nucleotides of the overhang are not base-paired. In one embodiment of the invention, the interfering RNA comprises a 3′ overhang of TT or UU. In another embodiment of the invention, the interfering RNA comprises at least one blunt end. The termini usually have a 5′ phosphate group or a 3′ hydroxyl group. In other embodiments, the antisense strand has a 5′ phosphate group, and the sense strand has a 5′ hydroxyl group. In still other embodiments, the termini are further modified by covalent addition of other molecules or functional groups.

The sense and antisense strands of the double-stranded siRNA may be in a duplex formation of two single strands as described above or may be a single-stranded molecule where the regions of complementarity are base-paired and are covalently linked by a linker molecule to form a hairpin loop when the regions are hybridized to each other. It is believed that the hairpin is cleaved intracellularly by a protein termed dicer to form an interfering RNA of two individual base-paired RNA molecules. A linker molecule can also be designed to comprise a restriction site that can be cleaved in vivo or in vitro by a particular nuclease.

In one embodiment, the invention provides an interfering RNA molecule that comprises a region of at least 13 contiguous nucleotides having at least 90% sequence complementarity to, or at least 90% sequence identity with, the penultimate 13 nucleotides of the 3′ end of an mRNA corresponding to a DNA target, which allows a one nucleotide substitution within the region. Two nucleotide substitutions (i.e., 11/13=85% identity/complementarity) are not included in such a phrase. In another embodiment, the invention provides an interfering RNA molecule that comprises a region of at least 14 contiguous nucleotides having at least 85% sequence complementarity to, or at least 85% sequence identity with, the penultimate 14 nucleotides of the 3′ end of an mRNA corresponding to a DNA target. Two nucleotide substitutions (i.e., 12/14=86% identity/complementarity) are included in such a phrase. In a further embodiment, the invention provides an interfering RNA molecule that comprises a region of at least 15, 16, 17, or 18 contiguous nucleotides having at least 80% sequence complementarity to, or at least 80% sequence identity with, the penultimate 14 nucleotides of the 3′ end of an mRNA corresponding to a DNA target. Three nucleotide substitutions are included in such a phrase.

The penultimate base in a nucleic acid sequence that is written in a 5′ to 3′ direction is the next to the last base, i.e., the base next to the 3′ base. The penultimate 13 bases of a nucleic acid sequence written in a 5′ to 3′ direction are the last 13 bases of a sequence next to the 3′ base and not including the 3′ base. Similarly, the penultimate 14, 15, 16, 17, or 18 bases of a nucleic acid sequence written in a 5′ to 3′ direction are the last 14, 15, 16, 17, or 18 bases of a sequence, respectively, next to the 3′ base and not including the 3′ base.

Interfering RNAs may be generated exogenously by chemical synthesis, by in vitro transcription, or by cleavage of longer double-stranded RNA with dicer or another appropriate nuclease with similar activity. Chemically synthesized interfering RNAs, produced from protected ribonucleoside phosphoramidites using a conventional DNA/RNA synthesizer, may be obtained from commercial suppliers such as Ambion Inc. (Austin, Tex.), Invitrogen (Carlsbad, Calif.), or Dharmacon (Lafayette, Colo.). Interfering RNAs can be purified by extraction with a solvent or resin, precipitation, electrophoresis, chromatography, or a combination thereof, for example. Alternatively, interfering RNA may be used with little if any purification to avoid losses due to sample processing.

When interfering RNAs are produced by chemical synthesis, phosphorylation at the 5′ position of the nucleotide at the 5′ end of one or both strands (when present) can enhance siRNA efficacy and specificity of the bound RISC complex, but is not required since phosphorylation can occur intracellularly.

Interfering RNAs can also be expressed endogenously from plasmid or viral expression vectors or from minimal expression cassettes, for example, PCR generated fragments comprising one or more promoters and an appropriate template or templates for the interfering RNA. Examples of commercially available plasmid-based expression vectors for shRNA include members of the pSilencer series (Ambion, Austin, Tex.) and pCpG-siRNA (InvivoGen, San Diego, Calif.). Viral vectors for expression of interfering RNA may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT™-DEST (Invitrogen, Carlsbad, Calif.). Selection of viral vectors, methods for expressing the interfering RNA from the vector and methods of delivering the viral vector are within the ordinary skill of one in the art. Examples of kits for production of PCR-generated shRNA expression cassettes include Silencer Express (Ambion, Austin, Tex.) and siXpress (Mirus, Madison, Wis.).

In certain embodiments, a first interfering RNA may be administered via in vivo expression from a first expression vector capable of expressing the first interfering RNA and a second interfering RNA may be administered via in vivo expression from a second expression vector capable of expressing the second interfering RNA, or both interfering RNAs may be administered via in vivo expression from a single expression vector capable of expressing both interfering RNAs. Additional interfering RNAs can be administered in a like manner (i.e. via separate expression vectors or via a single expression vector capable of expressing multiple interfering RNAs).

Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

In certain embodiments of the present invention, an antisense strand of an interfering RNA hybridizes with an mRNA in vivo as part of the RISC complex.

“Hybridization” refers to a process in which single-stranded nucleic acids with complementary or near-complementary base sequences interact to form hydrogen-bonded complexes called hybrids. Hybridization reactions are sensitive and selective. In vitro, the specificity of hybridization (i.e., stringency) is controlled by the concentrations of salt or formamide in prehybridization and hybridization solutions, for example, and by the hybridization temperature; such procedures are well known in the art. In particular, stringency is increased by reducing the concentration of salt, increasing the concentration of formamide, or raising the hybridization temperature.

For example, high stringency conditions could occur at about 50% formamide at 37° C. to 42° C. Reduced stringency conditions could occur at about 35% to 25% formamide at 30° C. to 35° C. Examples of stringency conditions for hybridization are provided in Sambrook, J., 1989, Molecular Cloning: A Laboratory Manual, Cold Spring Harbor Laboratory Press, Cold Spring Harbor, N.Y. Further examples of stringent hybridization conditions include 400 mM NaCl, 40 mM PIPES pH 6.4, 1 mM EDTA, 50° C. or 70° C. for 12-16 hours followed by washing, or hybridization at 70° C. in 1×SSC or 50° C. in 1×SSC, 50% formamide followed by washing at 70° C. in 0.3×SSC, or hybridization at 70° C. in 4×SSC or 50° C. in 4×SSC, 50% formamide followed by washing at 67° C. in 1×SSC. The temperature for hybridization is about 5-10° C. less than the melting temperature (Tₘ) of the hybrid where Tₘ is determined for hybrids between 19 and 49 base pairs in length using the following calculation: Tₘ ° C.=81.5+16.6(log₁₀[Na+])+0.41 (% G+C)−(600/N) where N is the number of bases in the hybrid, and [Na+] is the concentration of sodium ions in the hybridization buffer.

The above-described in vitro hybridization assay provides a method of predicting whether binding between a candidate siRNA and a target will have specificity. However, in the context of the RISC complex, specific cleavage of a target can also occur with an antisense strand that does not demonstrate high stringency for hybridization in vitro.

Interfering RNAs may differ from naturally-occurring RNA by the addition, deletion, substitution or modification of one or more nucleotides. Non-nucleotide material may be bound to the interfering RNA, either at the 5′ end, the 3′ end, or internally. Such modifications are commonly designed to increase the nuclease resistance of the interfering RNAs, to improve cellular uptake, to enhance cellular targeting, to assist in tracing the interfering RNA, to further improve stability, or to reduce the potential for activation of the interferon pathway. For example, interfering RNAs may comprise a purine nucleotide at the ends of overhangs. Conjugation of cholesterol to the 3′ end of the sense strand of an siRNA molecule by means of a pyrrolidine linker, for example, also provides stability to an siRNA.

Further modifications include a 3′ terminal biotin molecule, a peptide known to have cell-penetrating properties, a nanoparticle, a peptidomimetic, a fluorescent dye, or a dendrimer, for example.

Nucleotides may be modified on their base portion, on their sugar portion, or on the phosphate portion of the molecule and function in embodiments of the present invention. Modifications include substitutions with alkyl, alkoxy, amino, deaza, halo, hydroxyl, thiol groups, or a combination thereof, for example. Nucleotides may be substituted with analogs with greater stability such as replacing a ribonucleotide with a deoxyribonucleotide, or having sugar modifications such as 2′ OH groups replaced by 2′ amino groups, 2′ O-methyl groups, 2′ methoxyethyl groups, or a 2′-O, 4′-C methylene bridge, for example. Examples of a purine or pyrimidine analog of nucleotides include a xanthine, a hypoxanthine, an azapurine, a methylthioadenine, 7-deaza-adenosine and O- and N-modified nucleotides. The phosphate group of the nucleotide may be modified by substituting one or more of the oxygens of the phosphate group with nitrogen or with sulfur (phosphorothioates). Modifications are useful, for example, to enhance function, to improve stability or permeability, or to direct localization or targeting.

In certain embodiments, an interfering molecule of the invention comprises at least one of the modifications as described above.

In certain embodiments, the invention provides pharmaceutical compositions (also referred to herein as “compositions”) comprising an interfering RNA molecule of the invention. Pharmaceutical compositions are formulations that comprise interfering RNAs, or salts thereof, of the invention up to 99% by weight mixed with a physiologically acceptable carrier medium, including those described infra, and such as water, buffer, saline, glycine, hyaluronic acid, mannitol, and the like.

Interfering RNAs of the present invention are administered as solutions, suspensions, or emulsions. The following are examples of pharmaceutical composition formulations that may be used in the methods of the invention.

| | Amount in weight % |
|---|---|
| Interfering RNA | up to 99; 0.1-99; 0.1-50; |
| | 0.5-10.0 |
| Hydroxypropylmethylcellulose | 0.5 |
| Sodium chloride | 0.8 |
| Benzalkonium Chloride | 0.01 |
| EDTA | 0.01 |
| NaOH/HCl | qs pH 7.4 |
| Purified water (RNase-free) | qs 100 mL |

| | Amount in weight % |
|---|---|
| Interfering RNA | up to 99; 0.1-99; 0.1-50; |
| | 0.5-10.0 |
| Phosphate Buffered Saline | 1.0 |
| Benzalkonium Chloride | 0.01 |
| Polysorbate 80 | 0.5 |
| Purified water (RNase-free) | q.s. to 100% |

| | Amount in weight % |
|---|---|
| Interfering RNA | up to 99; 0.1-99; 0.1-50; |
| | 0.5-10.0 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Cremophor EL | 0.1 |
| Benzalkonium chloride | 0.01 |
| HCl and/or NaOH | pH 7.3-7.4 |
| Purified water (RNase-free) | q.s. to 100% |

| | Amount in weight % |
|---|---|
| Interfering RNA | up to 99; 0.1-99; 0.1-50; |
| | 0.5-10.0 |
| Phosphate Buffered Saline | 1.0 |
| Hydroxypropyl-β-cyclodextrin | 4.0 |
| Purified water (RNase-free) | q.s. to 100% |

As used herein the term “effective amount” refers to the amount of interfering RNA or a pharmaceutical composition comprising an interfering RNA determined to produce a therapeutic response in a mammal. Such therapeutically effective amounts are readily ascertained by one of ordinary skill in the art and using methods as described herein.

Generally, an effective amount of the interfering RNAs of the invention results in an extracellular concentration at the surface of the target cell of from 100 pM to 1 μM, or from 1 nM to 100 nM, or from 5 nM to about 50 nM, or to about 25 nM. The dose required to achieve this local concentration will vary depending on a number of factors including the delivery method, the site of delivery, the number of cell layers between the delivery site and the target cell or tissue, whether delivery is local or systemic, etc. The concentration at the delivery site may be considerably higher than it is at the surface of the target cell or tissue. Topical compositions can be delivered to the surface of the target organ, such as the eye, one to four times per day, or on an extended delivery schedule such as daily, weekly, bi-weekly, monthly, or longer, according to the routine discretion of a skilled clinician. The pH of the formulation is about pH 4.0 to about pH 9.0, or about pH 4.5 to about pH 7.4.

An effective amount of a formulation may depend on factors such as the age, race, and sex of the subject, the severity of the AMD, the rate of target gene transcript/protein turnover, the interfering RNA potency, and the interfering RNA stability, for example. In one embodiment, the interfering RNA is delivered topically to a target organ and reaches the HTRA1 mRNA-containing tissue such as the trabecular meshwork, retina or optic nerve head at a therapeutic dose thereby ameliorating HTRA1-associated disease process.

Therapeutic treatment of patients with interfering RNAs directed against HTRA1 mRNA is expected to be beneficial over small molecule treatments by increasing the duration of action, thereby allowing less frequent dosing and greater patient compliance, and by increasing target specificity, thereby reducing side effects.

An “acceptable carrier” as used herein refers to those carriers that cause at most, little to no ocular irritation, provide suitable preservation if needed, and deliver one or more interfering RNAs of the present invention in a homogenous dosage. An acceptable carrier for administration of interfering RNA of embodiments of the present invention include the cationic lipid-based transfection reagents TransIT® -TKO (Mirus Corporation, Madison, Wis.), LIPOFECTIN®, Lipofectamine, OLIGOFECTAMINE™ (Invitrogen, Carlsbad, Calif.), or DHARMAFECT™ (Dharmacon, Lafayette, Colo.); polycations such as polyethyleneimine; cationic peptides such as Tat, polyarginine, or Penetratin (Antp peptide); nanoparticles; or liposomes. Liposomes are formed from standard vesicle-forming lipids and a sterol, such as cholesterol, and may include a targeting molecule such as a monoclonal antibody having binding affinity for cell surface antigens, for example. Further, the liposomes may be PEGylated liposomes.

The interfering RNAs may be delivered in solution, in suspension, or in bioerodible or non-bioerodible delivery devices. The interfering RNAs can be delivered alone or as components of defined, covalent conjugates. The interfering RNAs can also be complexed with cationic lipids, cationic peptides, or cationic polymers; complexed with proteins, fusion proteins, or protein domains with nucleic acid binding properties (e.g., protamine); or encapsulated in nanoparticles or liposomes. Tissue- or cell-specific delivery can be accomplished by the inclusion of an appropriate targeting moiety such as an antibody or antibody fragment.

Interfering RNA may be delivered via aerosol, buccal, dermal, intradermal, inhaling, intramuscular, intranasal, intraocular, intrapulmonary, intravenous, intraperitoneal, nasal, ocular, oral, otic, parenteral, patch, subcutaneous, sublingual, topical, or transdermal administration, for example.

In certain embodiments, treatment of ocular disorders with interfering RNA molecules is accomplished by administration of an interfering RNA molecule directly to the eye. Local administration to the eye is advantageous for a number or reasons, including: the dose can be smaller than for systemic delivery, and there is less chance of the molecules silencing the gene target in tissues other than in the eye.

A number of studies have shown successful and effective in vivo delivery of interfering RNA molecules to the eye. For example, Kim et al. demonstrated that subconjunctival injection and systemic delivery of siRNAs targeting VEGF pathway genes inhibited angiogenesis in a mouse eye (Kim et al., 2004, Am. J. Pathol. 165:2177-2185). In addition, studies have shown that siRNA delivered to the vitreous cavity can diffuse throughout the eye, and is detectable up to five days after injection (Campochiaro, 2006, Gene Therapy 13:559-562).

Interfering RNA may be delivered directly to the eye by ocular tissue injection such as periocular, conjunctival, subtenon, intracameral, intravitreal, intraocular, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

For ophthalmic delivery, an interfering RNA may be combined with ophthalmologically acceptable preservatives, co-solvents, surfactants, viscosity enhancers, penetration enhancers, buffers, sodium chloride, or water to form an aqueous, sterile ophthalmic suspension or solution. Solution formulations may be prepared by dissolving the interfering RNA in a physiologically acceptable isotonic aqueous buffer. Further, the solution may include an acceptable surfactant to assist in dissolving the interfering RNA. Viscosity building agents, such as hydroxymethyl cellulose, hydroxyethyl cellulose, methylcellulose, polyvinylpyrrolidone, or the like may be added to the compositions of the present invention to improve the retention of the compound.

In order to prepare a sterile ophthalmic ointment formulation, the interfering RNA is combined with a preservative in an appropriate vehicle, such as mineral oil, liquid lanolin, or white petrolatum. Sterile ophthalmic gel formulations may be prepared by suspending the interfering RNA in a hydrophilic base prepared from the combination of, for example, CARBOPOL®-940 (BF Goodrich, Charlotte, N.C.), or the like, according to methods known in the art. VISCOAT® (Alcon Laboratories, Inc., Fort Worth, Tex.) may be used for intraocular injection, for example. Other compositions of the present invention may contain penetration enhancing agents such as cremephor and TWEEN® 80 (polyoxyethylene sorbitan monolaureate, Sigma Aldrich, St. Louis, Mo.), in the event the interfering RNA is less penetrating in the eye.

In certain embodiments, the invention also provides a kit that includes reagents for attenuating the expression of an mRNA as cited herein in a cell. The kit contains an siRNA or an shRNA expression vector. For siRNAs and non-viral shRNA expression vectors the kit also contains a transfection reagent or other suitable delivery vehicle. For viral shRNA expression vectors, the kit may contain the viral vector and/or the necessary components for viral vector production (e.g., a packaging cell line as well as a vector comprising the viral vector template and additional helper vectors for packaging). The kit may also contain positive and negative control siRNAs or shRNA expression vectors (e.g., a non-targeting control siRNA or an siRNA that targets an unrelated mRNA). The kit also may contain reagents for assessing knockdown of the intended target gene (e.g., primers and probes for quantitative PCR to detect the target mRNA and/or antibodies against the corresponding protein for western blots). Alternatively, the kit may comprise an siRNA sequence or an shRNA sequence and the instructions and materials necessary to generate the siRNA by in vitro transcription or to construct an shRNA expression vector.

A pharmaceutical combination in kit form is further provided that includes, in packaged combination, a carrier means adapted to receive a container means in close confinement therewith and a first container means including an interfering RNA composition and an acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

The references cited herein, to the extent that they provide exemplary procedural or other details supplementary to those set forth herein, are specifically incorporated by reference.

Those of skill in the art, in light of the present disclosure, will appreciate that obvious modifications of the embodiments disclosed herein can be made without departing from the spirit and scope of the invention. All of the embodiments disclosed herein can be made and executed without undue experimentation in light of the present disclosure. The full scope of the invention is set out in the disclosure and equivalent embodiments thereof. The specification should not be construed to unduly narrow the full scope of protection to which the present invention is entitled.

While a particular embodiment of the invention has been shown and described, numerous variations and alternate embodiments will occur to those skilled in the art. Accordingly, the invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes to the claims that come within the meaning and range of equivalency of the claims are to be embraced within their scope. Further, all published documents, patents, and applications mentioned herein are hereby incorporated by reference, as if presented in their entirety.

### EXAMPLES

The following example, including the experiments conducted and results achieved are provided for illustrative purposes only and are not to be construed as limiting the invention.

### Example 1

### Interfering RNA for Specifically Silencing HTRA1 in HeLa Cells

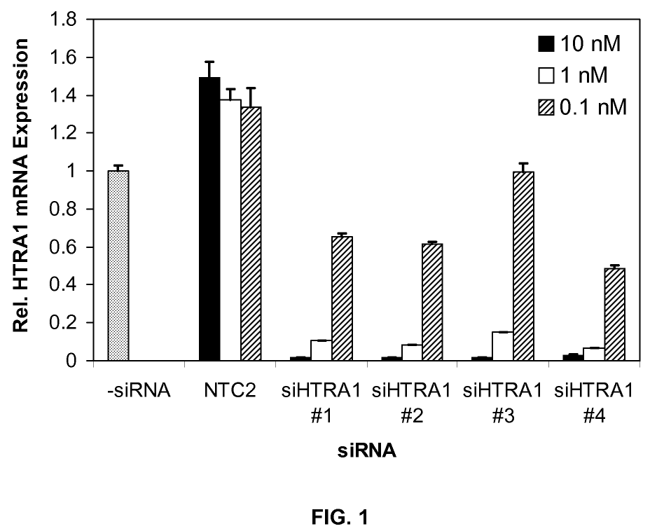

To study the ability of HTRA1 interfering RNA to knock down the levels of HTRA1 mRNA expression in cultured HeLa cells, transfection of HeLa cells was accomplished using standard in vitro concentrations (0.1-10 nM) of HTRA1 siRNAs or siCONTROL Non-targeting siRNA #2 (NTC2) and DHARMAFECT® #1 transfection reagent (Dharmacon, Lafayette, Colo.). All siRNAs were dissolved in 1× siRNA buffer, an aqueous solution of 20 mM KCl, 6 mM HEPES (pH 7.5), 0.2 mM MgCl₂. Control samples included a buffer control in which the volume of siRNA was replaced with an equal volume of 1× siRNA buffer (-siRNA). HTRA1 mRNA level was determined by qRT-PCR using High Capacity cDNA Reverse Transcription Kit, Assays-On-Demand Gene Expression kits, TaqMan Universal PCR Master Mix, and an ABI PRISM 7700 Sequence Detector (Applied Biosystems, Foster City, Calif.). HTRA1 mRNA expression was normalized to β-actin mRNA level, and reported relative to HTRA1 expression in non-transfected cells (-siRNA). The HTRA1 siRNAs are double-stranded interfering RNAs having specificity for the following targets: siHTRA1 #1 was derived from SEQ ID NO: 31; siHTRA1 #2 was derived from SEQ ID NO: 41; siHTRA1 #3 was derived from SEQ ID NO: 70; siHTRA1 #4 was derived from SEQ ID NO: 22. As shown in FIG. 1, transfection with all four of the siHTRA1 siRNAs reduced HTRA1 mRNA expression significantly (>70% relative to the -siRNA control) at the 10 and 1 nM concentrations, but exhibited reduced efficacy at 0.1 nM. The siHTRA1 #4 siRNA was particularly effective.

It should be understood that the foregoing disclosure emphasizes certain specific embodiments of the invention and that all modifications or alternatives equivalent thereto are within the spirit and scope of the invention as set forth in the appended claims.

What is claimed is:

1. A method of attenuating expression of a HTRA1 mRNA in an eye of a patient, comprising administering to the eye of the patient an interfering RNA molecule that down regulates expression of the HTRA1 mRNA via RNA interference, wherein the interfering RNA molecule is administered via ocular injection.

2. The method of claim 1, wherein the interfering RNA molecule is double stranded and each strand is independently about 19 to about 27 nucleotides in length.

3. The method of claim 2, wherein each strand is independently about 19 nucleotides to about 25 nucleotides in length.

4. The method of claim 2, wherein each strand is independently about 19 nucleotides to about 21 nucleotides in length.

5. The method of claim 2, wherein the sense and antisense strands are connected by a linker to form an shRNA that can attenuate expression of HTRA1 mRNA in a patient.

6. The method of claim 2, wherein the interfering RNA molecule has blunt ends.

7. The method of claim 2, wherein at least one strand of the interfering RNA molecule comprises a 3′ overhang.

8. The method of claim 7, wherein the 3′ overhang comprises about 1 to about 6 nucleotides.

9. The method of claim 8, wherein the 3′ overhang comprises 2 nucleotides.

10. The method of claim 1, wherein the interfering RNA molecule is administered via in vivo expression from an expression vector capable of expressing the interfering RNA molecule.

11. The method of claim 1, wherein the patient has or is at risk of developing an HTRA1 -mediated ocular disorder.

12. The method of claim 11, wherein the HTRA1-mediated ocular disorder is wet or dry age-related macular degeneration.

13. The method of claim 1, wherein the interfering RNA molecule recognizes a portion of HTRA1 mRNA that corresponds to any of SEQ ID NO: 2-110.

14. The method of claim 1, wherein the interfering RNA molecule recognizes a portion of HTRA1 mRNA, wherein the portion comprises nucleotide 604, 612, 613, 614, 615, 616, 617, 618, 619, 620, 621, 622, 623, 624, 634, 643, 646, 671, 676, 731, 732, 733, 734, 738, 742, 745, 759, 761, 763, 765, 796, 797, 799, 800, 802, 808, 810, 811, 823, 824, 826, 827, 833, 850, 853, 854, 857, 873, 874, 875, 892, 1057, 1060, 1081, 1084, 1123, 1130, 1135, 1136, 1137, 1138, 1162, 1163, 1173, 1186, 1234, 1235, 1248, 1249, 1252, 1258, 1265, 1272, 1300, 1306, 1312, 1349, 1351, 1354, 1360, 1401, 1402, 1414, 1422, 1423, 1424, 1468, 1469, 1489, 1514, 1531, 1543, 1544, 1552, 1577, 1721, 1847, 1848, 1872, 1873, 1874, 1875, 1887, 1898, 1900, 1904, 2082, 2083, or 2084 of SEQ ID NO: 1.

15. The method of claim 1, wherein the interfering RNA molecule is administered via a topical, intravitreal, transcleral, conjunctival, subtenon, intracameral, subretinal, subconjunctival, or intracanalicular route.

16. The method of claim 1, wherein the interfering RNA molecule comprises at least one modification.

17. The composition of claim 1, wherein the interfering RNA molecule is a shRNA, a siRNA, or a miRNA.

18. An interfering RNA molecule having a length of about 19 to about 49 nucleotides, the interfering RNA molecule comprising:
(a) a region of at least 13 contiguous nucleotides having at least 90% sequence complementarity to, or at least 90% sequence identity with, the penultimate 13 nucleotides of the 3′ end of a mRNA corresponding to any one of SEQ ID NO: 2-110;
(b) a region of at least 14 contiguous nucleotides having at least 85% sequence complementarity to, or at least 85% sequence identity with, the penultimate 14 nucleotides of the 3′ end of an mRNA corresponding to any one of SEQ ID NO: 2-110; or
(c) a region of at least 15, 16, 17, or 18 contiguous nucleotides having at least 80% sequence complementarity to, or at least 80% sequence identity with, the penultimate 15, 16, 17, or 18 nucleotides, respectively, of the 3′ end of an mRNA corresponding to any one of SEQ ID NO: 2-110.

19. The interfering RNA molecule of claim 18, wherein the interfering RNA molecule recognizes a portion of HTRA1 mRNA that corresponds to any of SEQ ID NO: 2-110.

20. The interfering RNA molecule of claim 18, wherein the interfering RNA molecule recognizes a portion of HTRA1 mRNA, wherein the portion comprises nucleotide 604, 612, 613, 614, 615, 616, 617, 618, 619, 620, 621, 622, 623, 624, 634, 643, 646, 671, 676, 731, 732, 733, 734, 738, 742, 745, 759, 761, 763, 765, 796, 797, 799, 800, 802, 808, 810, 811, 823, 824, 826, 827, 833, 850, 853, 854, 857, 873, 874, 875, 892, 1057, 1060, 1081, 1084, 1123, 1130, 1135, 1136, 1137, 1138, 1162, 1163, 1173, 1186, 1234, 1235, 1248, 1249, 1252, 1258, 1265, 1272, 1300, 1306, 1312, 1349, 1351, 1354, 1360, 1401, 1402, 1414, 1422, 1423, 1424, 1468, 1469, 1489, 1514, 1531, 1543, 1544, 1552, 1577, 1721, 1847, 1848, 1872, 1873, 1874, 1875, 1887, 1898, 1900, 1904, 2082, 2083, or 2084.

21. The interfering RNA molecule of claim 18, wherein the interfering RNA molecule is a shRNA, a siRNA, or a miRNA.

22. The interfering RNA molecule of claim 18, wherein the interfering RNA molecule comprises at least one modification.

23. The interfering RNA molecule of claim 18, wherein the interfering RNA molecule is double stranded, and wherein at least one strand of the interfering RNA molecule comprises a 3′ overhang.

24. The interfering RNA molecule of claim 23, wherein the 3′ overhang comprises about 1 to about 6 nucleotides.

25. The interfering RNA molecule of claim 23, wherein the 3′ overhang comprises 2 nucleotides.

26. The interfering RNA molecule of claim 18, wherein the interfering RNA molecule is double stranded, and the interfering RNA molecule has blunt ends.

27. A method of treating an HTRA1-mediated ocular disorder in a patient in need thereof, comprising administering to the patient the interfering RNA molecule of claim 18, wherein the interfering RNA molecule is administered by ocular injection.

28. The method of claim 27, wherein the patient has or is at risk of developing an HTRA1-mediated ocular disorder.

29. The method of claim 28, wherein the HTRA1-mediated ocular disorder is wet or dry age-related macular degeneration.

30. The method of claim 27, wherein the interfering RNA molecule is administered via in vivo expression from an interfering RNA molecule expression vector.
